(19) United States
(12) Patent Application Publication
(10) Pub. No.: US 2011/0219508 A1
(43) Pub. Date: Sep. 15, 2011

(54) Adhesive Patch for Securing Eyewear to Headwear

(76) Inventor: John D. Hill, Collbran, CO (US)

(21) Appl. No.: 12/721,370

(22) Filed: Mar. 10, 2010

(51) Int. Cl. A61F 9/00; C09J 7/00; A42B 1/24

(52) U.S. Cl. 2/10; 428/343; 2/209.13

(57) ABSTRACT

Described herein is an apparatus for securing eyewear to headwear. The apparatus includes a plurality of patches. Each of the plurality of patches comprise an elastomeric polymer configured to frictionally cling to a stem of the eyewear when the stem of the eyewear is placed on or over the surface. At least a first patch of the plurality of patches is disposed on a first outer side of the headwear and at least a second patch of the plurality of patches is disposed on a second outer side of the headwear. An adhesive is disposed at least partially on an underside of each of the plurality of patches. The adhesive is configured to secure each of the plurality of patches to the at least the first outer side of the headwear and the second outer side of the headwear respectively.

### TECHNICAL FIELD

The present disclosure is generally related to headwear.

### BACKGROUND

It is common practice for individuals to wear sunglasses, reading glasses or other types of eyewear. Sunglasses are commonly used in the outdoors to protect the individual's eyes against the harmful rays of the sun or to enable an individual to see better in bright sunlight. It is also common practice for individuals to further utilize headwear, such as hats, caps, visors or headbands, for example, to provide further protection against the sun or to keep sweat out of the individual's face. It often becomes desirable to remove eyewear, such as sunglasses, when entering an environment that is not as bright as the outdoors.

### BRIEF DESCRIPTION OF THE DRAWINGS

FIG. 1A is an isometric view of a patch according to embodiments.

FIG. 1B is a side view of the patch of FIG. 1A according to embodiments.

FIG. 2A is an isometric view of a patch coupled to a first side of headwear according to embodiments.

FIG. 2B is an isometric view of a position of a patch on a second side of headwear according to embodiments.

FIG. 3A is an isometric view of eyewear positioned on headwear with a patch securing the eyewear to the headwear according to embodiments.

FIG. 3B is an isometric view of eyewear positioned on headwear according to alternative embodiments.

FIG. 4 is a flow chart depicting a method for securing eyewear to headwear according to embodiments.

### DETAILED DESCRIPTION

This disclosure will now more fully describe exemplary embodiments with reference to the accompanying drawings, in which specific embodiments are shown. Other aspects may be embodied in many different forms and the inclusion of specific embodiments in the disclosure should not be construed as limiting such aspects to the embodiments set forth herein. Rather, the embodiments depicted in the drawings are included to provide a disclosure that is thorough and complete and which fully conveys the intended scope to those skilled in the art. When referring to the figures, like structures and elements are shown throughout are indicated with like reference numerals.

### Terminology

The terms and phrases as indicated in quotes (“ ”) in this section are intended to have the meaning ascribed to them in this Terminology section applied to them throughout this document including the claims unless clearly indicated otherwise in context. Further, as applicable, the stated definitions are to apply, regardless of the word or phrase's case, to the singular and plural variations of the defined word or phrase.

The term “or” as used in this specification and the appended claims is not meant to be exclusive rather the term is inclusive meaning “either or both”.

References in the specification to “one embodiment”, “an embodiment”, “a preferred embodiment”, “an alternative embodiment” and similar phrases mean that a particular feature, structure, or characteristic described in connection with the embodiment is included in at least an embodiment of the invention. The appearances of the phrase “in one embodiment” in various places in the specification are not necessarily all meant to refer to the same embodiment.

The term “couple” or “coupled” as used in this specification and the appended claims refers to either an indirect or direct connection between the identified elements, components or objects. Often the manner of the coupling will be related specifically to the manner in which the two coupled elements interact.

The term “eyewear” as used herein means sunglasses, eyeglasses, reading glasses and any other article that may be used to assist a user is covering or protecting they eyes and/or assisting the user to see.

The term “headwear” as used herein means any article of clothing that is placed on a head of an individual.

Directional and/or relationary terms such as, but not limited to, left, right, nadir, apex, top, bottom, vertical, horizontal, back, front and lateral are relative to each other and are dependent on the specific orientation of an applicable element or article, and are used accordingly to aid in the description of the various embodiments and are not necessarily intended to be construed as limiting.

Described herein is an apparatus for securing eyewear to headwear. The apparatus includes a plurality of patches. Each of the plurality of patches comprises an elastomeric polymer configured to prevent a stem of the eyewear from sliding across the surface of each of the plurality of patches when the stem of the eyewear is placed on or over the surface. At least a first patch of the plurality of patches is disposed on a first outer side of the headwear and at least a second patch of the plurality of patches is disposed on a second outer side of the headwear. An adhesive is disposed at least partially on an underside of each of the plurality of patches. The adhesive is configured to secure each of the plurality of patches to the at least the first outer side of the headwear and the second outer side of the headwear respectively.

Also described herein is a method of securing eyewear to headwear. According to the method, a first patch is adhered on a first side of the headwear and a second patch is adhered to a second side of the headwear. The first patch and the second patch comprise an elastomeric polymer. The method also includes placing a first portion of the eyewear on a surface of the first patch and placing a second portion of the eyewear on a surface of the second patch. When the first portion of the eyewear is on the surface of the first patch and the second portion of the eyewear is on the surface of the second patch, the first and second portions of the eyewear frictionally cling to the first and second patch respectively.

Still yet other embodiments provide for a patch comprising a first layer and a second layer. The first layer comprises an adhesive configured to secure the patch to a side of headwear and the second layer is comprised of an elastomeric polymer configured to frictionally cling to an article when the article is placed on the surface thereof.

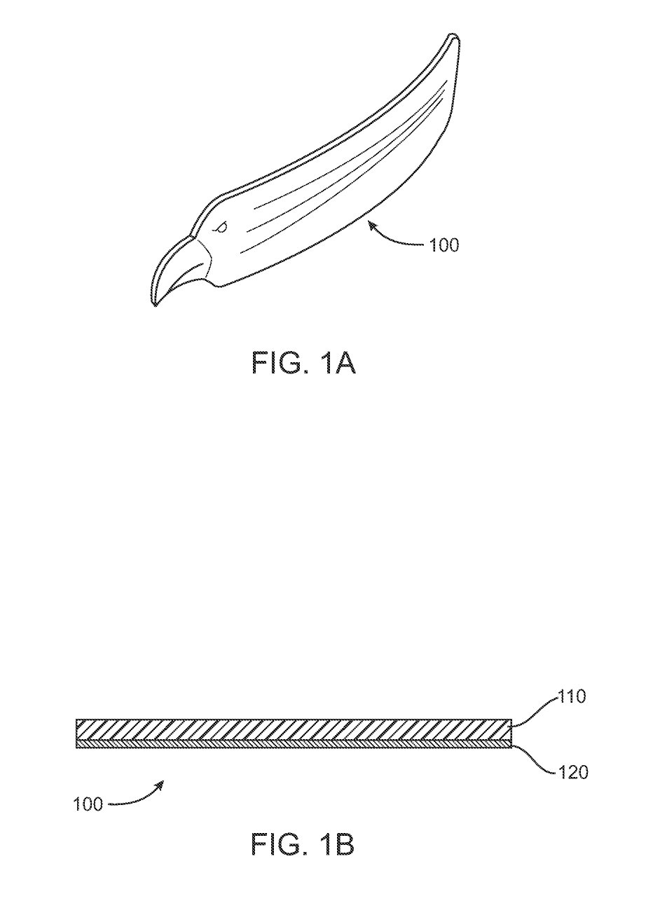

FIG. 1A is an isometric view of a patch 100 according to embodiments. The patch 100 may be comprised of an elastomeric polymer, such as, for example a polyurethane material. The polyurethane material provides a frictional or tacky quality that remains disposed on the surface and is transparent or translucent. In addition, the surface of the patch may be cleaned by soap and water and still retain the frictional property. Other embodiments provide that a low adhesive substance may be placed on the surface of the patch 100 to further increase the hold of the patch. Other embodiments provide that the patch may be made of other materials such as a cloth based material.

The patch 100 may also be formed of or can include an expanded vinyl material. The vinyl material may provide a good frictional or tacky quality that remains disposed on the surface and that retains items that have been placed thereon. The expanded vinyl material of the patch forms a temporary non-chemical bond with the item placed thereon. Furthermore, the item may be removed from the surface of the patch without damaging the patch or the item.

More specifically, the expanded vinyl material may include diisodecy/phthalate, polymeric plasticer, a UV stabilizer, a vinyl hear stabilizer, a blowing agent for vinyl plastisol and vinyl resin.

Further, other embodiments of the patch 100 may comprise other polymeric materials have a suitably tacky surface (or surface having a high coefficient of friction) that tend to grip items in which they come in contact with. Examples of items that utilize suitably tacky materials include but are not limited to cell phone dash pads and receiver gloves often used by football players. An example of a suitable material is the material used by Reebok International Ltd. under the trademark Griptonite.

In embodiments, the surface of the patch may include a plurality of small bumps or raised portions. Such raised portions may increase the frictional property of the surface. Alternatively, the surface of the patch 100 may be smooth. Still yet other embodiments provide that the surface of the patch has small indentations dispersed on the surface.

As shown in FIG. 1A, the patch may be in the shape of a logo. Such examples include a logo of a professional sports team. The professional sports team may includes teams from professional organizations such as the NFL, NBA, NHL, WNBA, MLS, and the MLB (e.g., Seattle Seahawks, Denver Nuggets, Los Angeles Dodgers, Colorado Avalanche etc.) to name a few. It is also contemplated that the patch 100 may be a car number in NASCAR (e.g., #2, #14 etc.). Other embodiments provide that the logo may be in the shape of a semi-professional sports team, high school team, community team and the like. It is also contemplated that the logo 100 may be in the shape of, and have corresponding colors to, a helmet or hat of a sports team (e.g. orange and brown helmet of the Cleveland Browns). The logo 100 may also be in the shape of a logo of a brand of item such as, for example, the Nike swoosh. Still yet other embodiments provide that the patch 100 is three-dimensional or has three dimensional aspects.

The patch 100 may also have varying lengths and widths depending on the logo used. For example, a patch with a logo of the Seattle Seahawks may have a longer length but a smaller width than a patch depicting a logo of the New York Yankees. Other embodiments provide that the length and/or width of the patch is uniform regardless of the logo used.

FIG. 1B is a side view of the patch 100 of FIG. 1A according to embodiments. The patch 100 may comprise multiple layers. The layers include an upper layer 110 and a lower layer 120. In embodiments, the upper layer 110 comprises the actual design of the logo while the bottom layer 120 is an adhesive layer. The upper layer 110 may also be the polyurethane layer or other material layer on which items are placed.

Embodiments provide that alternative means may be used to secure the patch 100 to headwear. Examples include, but are not limited to, stitching, pins, staples, rivets, and clips or other suitable mechanical fastening means.

It is also contemplated that the patch 100 may have multiple layers to give the patch 100 a three-dimensional effect. In such embodiments, the lower layer 120 may be a base support portion while the upper portion 110 is a raised, three-dimensional portion. As with other configurations, an adhesive layer may be disposed beneath the lower layer 120. Still yet other embodiments provide the patch 100 may be three-dimensional based on different layers of the actual logo. For example, the lower layer 120 may show a first part of the logo, while the upper layer 110 shows a second portion of the logo and only partially covers the lower layer 120. Thus, at least a portion of the lower layer may be visible. It is also contemplated that the upper layer 110 may be transparent or translucent. It is also contemplated that the patch itself may be molded into a three-dimensional shape or logo.

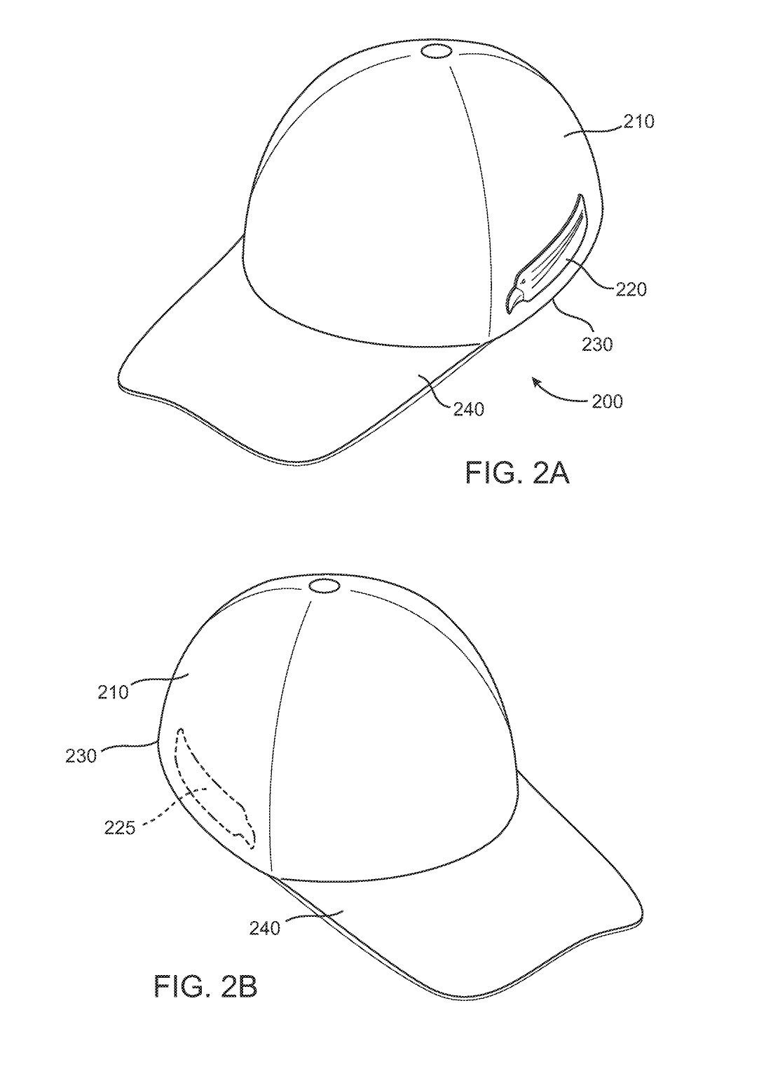

FIGS. 2A-4 illustrate various aspects and features of an apparatus, such as patch 100 (FIGS. 1A-1B) that may be used for securing eyewear to headwear according to embodiments. FIGS. 2A-4 may also show or use aspects of the patch 100 that were first described with respect to FIGS. 1A-1B. Therefore the description of FIGS. 2A-4 may refer to the patch 100 that described in FIGS. 1A-1B. However, any references to the patch 100 of FIGS. 1A-1B are for descriptive purposes only.

FIG. 2A shows an isometric view of patch and headwear combination 200 according to embodiments. The patch and headwear combination 200 consists of headwear 210 and a patch 220. According to embodiments, patch 220 may be similar to patch 100 of FIG. 1A.

According to embodiments, headwear 210 may consist of a baseball type cap, headband, beret, stocking cap, or any other article that may be placed on a head of an individual. As shown in FIG. 2A, a patch 220 is placed on a first side of the headwear 210. In embodiments where an adhesive layer is present on the underside of the patch 220, it is possible that the patch 220 may be placed on headwear 210 and subsequently removed. Additionally, the patch 220 may be moved to a different location on the headwear 210 and secured on the new location.

The patch 220 is placed on a first side of the headwear 210 near a crown portion 230 of the headwear 210. The patch 220 may also be adhered or otherwise secured, to the headwear so the entire patch 220 is placed on the headwear behind the bill portion 240 (if present) of the headwear 210. Other embodiments provide that at least a portion of the patch 220 is positioned near the crown portion 230 and behind the bill portion 240 of the headwear 210. Still yet other embodiments provide that the patch 220 may be placed on any portion of the headwear 210, so long as when eyewear is placed on the headwear 210, at least a portion of the eyewear is placed on or over the surface of the patch 220. When an item, such as the portion of the eyewear is placed on the surface of the patch, a non-chemical bond is formed between the surface of the patch and the portion of the eyewear. The non-chemical bond causes the patch to frictionally cling to any item that is placed thereon.

FIG. 2B is an isometric view of a position of a patch 235 on a second side of headwear 210 according to embodiments. A patch 220 may be placed on first and second sides of the headwear 210. As shown in FIG. 2B, a second patch may be placed opposite the patch 220 (FIG. 2A) that is coupled to the first side of the headwear 210. Such a configuration is shown by patch placement area 225. Having patches on either side of the headwear 220 may cause greater friction on portions of eyewear that area placed over or on each of the patches.

As with patch 220 located on the first side of the headwear 220, the second patch may be placed near the crown 230 of the headwear and be entirely or partially located behind the bill 240 (if present) on the headwear 210.

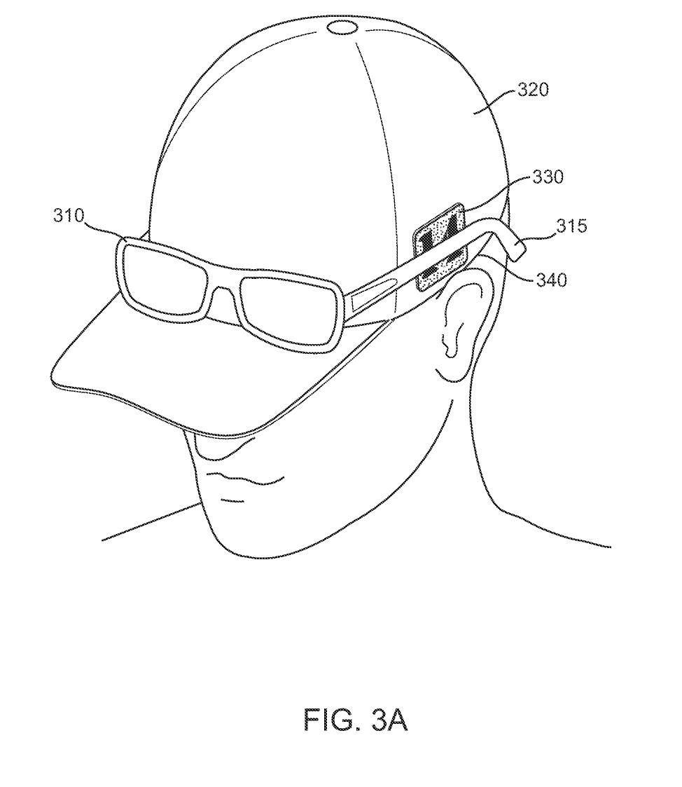

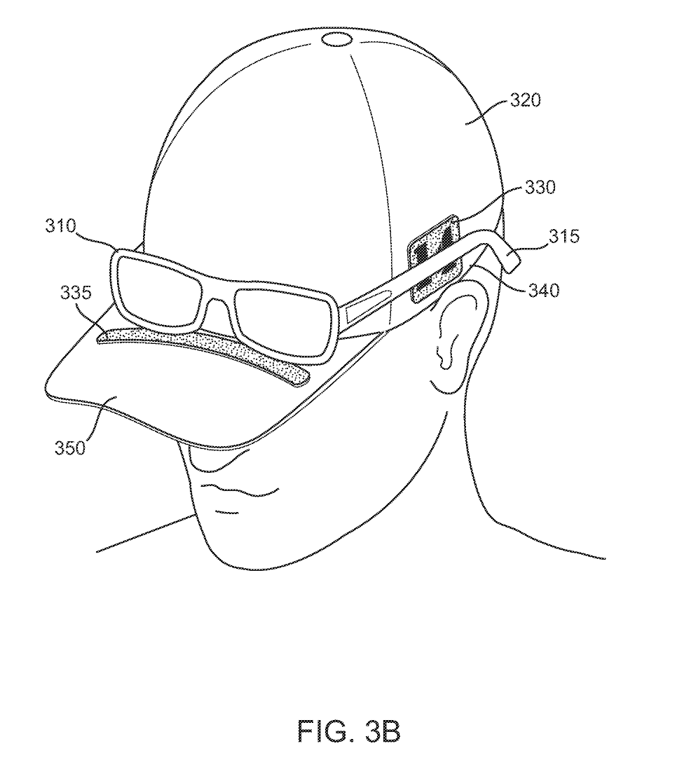

FIG. 3A is an isometric view of eyewear 310 positioned on headwear 320 with a patch 330 securing the eyewear 310 to the headwear 320 according to embodiments. Examples of eyewear include, but are not limited to sunglasses, eyeglasses, and reading glasses.

In embodiments, a patch 330 is secured to a first side of the headwear 320. Embodiments provide that a single patch 330 may be secured to one side of the headwear 320. It is also contemplated that a second patch may be positioned on a second side of the headwear 320, opposite the first patch on the first side of the headwear 320. The patch 330 may be placed on the headwear 320 near the crown portion 340 of the headwear 320.

When the headwear 320 is a on a head of an individual as shown in FIG. 3A, the patch may be positioned on the headwear so as to be above an ear of the individual. Although the patch 330 is shown to be above the ear of the individual, it is anticipated that the patch may be positioned anywhere on the headwear 320. For example, the patch may be placed on the headwear 320 so as to be located between a temple and an ear of an individual when the headwear 320 is placed on the individual's head.

As stated above, a stem 315 of the eyewear 310 is placed on a surface of the patch 330. When the stem 315 of the eyewear 310 is placed on the patch, movement of the stem 315 in an upward, forward, backward, downward motion, and any combination thereof, is restricted due to the increased friction caused by the surface of the patch 330. As a result, movement of the eyewear 310 is also restricted. Embodiments provide that a second stem (not shown) of the eyewear may be placed on a second patch on the opposite side of the patch on the first side of the eyewear. Thus, both stems of the eyewear 310 may be placed over a surface of the patch to more securely hold the eyewear when it is placed on the headwear.

FIG. 3B is an isometric view of eyewear 310 positioned on headwear 320 according to alternative embodiments. In addition to a patch being positioned on first and/or second sides of the headwear 320 as previously described, a third patch 335 may be placed on a frontal portion of the headwear 320. In instances where the headwear is hat with a bill 350, the third patch 335 may be placed on a top surface of the bill 350.

In embodiments, the third patch 335 is comprised of the same material as patch 100 (FIG. 1A). The third patch 335 may also be in the shape of a logo or have a team name, team colors etc. disposed thereon.

When placed on a front portion of headwear, a portion of the eyewear 310, such as, for example, the frames of the eyewear, 310 may be placed on or behind the third patch 335. Thus, movement of the eyewear may be further restricted as more surface area of the eyewear is disposed on various surfaces of patches configured to restrict movement of objects that have been placed thereon.

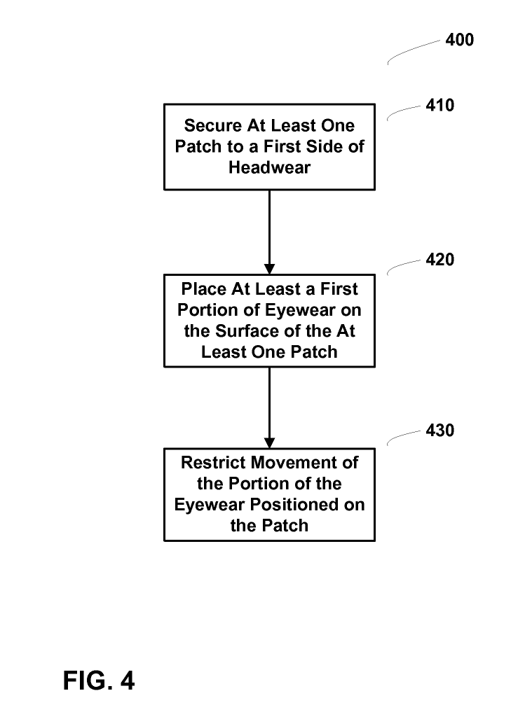

FIG. 4 is a flow chart depicting a method 400 for securing eyewear to headwear according to embodiments. The method 400 may be used to secure a patch, such as patch 100 (FIG. 1) to various types of headwear. Method 400 begins at step 410 in which at least one patch is secured to a first side of the headwear. The patch may be secured to the headwear via an adhesive layer that is disposed on an underside of the headwear. It is contemplated that other means of securing the patch may also be used.

In embodiments, the patch is made of an elastomeric polymer, such as, for example a polyurethane type of material. As described above, the surface of the patch has a tacky quality which causes increased friction between the surface of the patch and items placed thereon. It is also contemplated that the patch may be made of other materials while still providing increased friction.

The patch may be placed on first and/or second sides of the headwear. Embodiments provides that the patch may be placed on the headwear where desired by the individual. Other embodiments provide that the patch is placed on the headwear near a crown portion of the headwear. Regardless of the position of the patch on the headwear, the patch may be placed in a position so as to enable at least a portion of eyewear to rest on a surface of the patch when the eyewear is positioned on the headwear.

Step 420 provides that at least a first portion of eyewear is placed on the surface of the at least one patch. In embodiments, the portion of the eyewear is a stem portion of the eyewear. Other embodiments provide the portion of the eyewear is a frame portion of the eyewear. Still yet other embodiments provide that both stem and frame portions may be placed on a surface of a single patch or multiple patches.

Once at least a portion of the eyewear is placed on the surface of the patch, movement of the eyewear is restricted in step 430. Movement of the eyewear is restricted due to increased friction between the surface of the patch and the portion of the eyewear.

Although illustrative embodiments of the invention have been described in detail herein with reference to the accompanying drawings, it is to be understood that the invention is not limited to those precise embodiments. As such, many modifications and variations will be apparent to practitioners skilled in this art. Accordingly, it is intended that the scope of the invention be defined by the following claims and their equivalents. Furthermore, it is contemplated that a particular feature described either individually or as part of an embodiment can be combined with other individually described features, or parts of other embodiments, even if the other features and embodiments make no mentioned of the particular feature. Thus, the absence of describing combinations should not preclude the inventor from claiming rights to such combinations.

What is claimed is:

1. An apparatus for securing eyewear to headwear, the apparatus comprising:
a plurality of patches, wherein a surface of each of the plurality of patches comprise an elastomeric polymer configured to frictionally cling to a stem of the eyewear when the stem of the eyewear is placed thereon and wherein at least a first patch of the plurality of patches is disposed on a first outer side of the headwear and at least a second patch of the plurality of patches is disposed on a second outer side of the headwear;
an adhesive disposed at least partially on an underside of each of the plurality of patches, wherein the adhesive is configured to secure each of the plurality of patches to the at least the first outer side of the headwear and the second outer side of the headwear respectively.

2. The apparatus of claim 1, wherein each of the plurality of patches are in the shape of a logo.

3. The apparatus of claim 2, wherein the logo is a professional sports team logo.

4. The apparatus of claim 2, wherein the logo is a brand logo.

5. The apparatus of claim 1, wherein each of the patches are placed on the headwear so as to be positioned above ears of a user when the headwear is placed on a head of the user.

6. The apparatus of claim 1, wherein the each of the plurality of patches have at least two layers.

7. A method of securing eyewear to headwear, the method comprising:
adhering a first patch on a first side of the headwear and adhering a second patch on a second side of the headwear, wherein the first patch and the second patch comprise an elastomeric polymer; and
placing a first portion of the eyewear on a surface of the first patch and placing a second portion of the eyewear on a surface of the second patch, wherein when the first portion of the eyewear is on the surface of the first patch and the second portion of the eyewear is on the surface of the second patch, movement of each of the first portion of the eyewear and the second portion of the eyewear is restricted.

8. The method of claim 7, wherein at least one of the first patch and the second patch is in the shape of a logo.

9. The method of claim 7, wherein the first patch and the second patch have multiple layers.

10. The method of claim 7, wherein at least one layer of the multiple layers is an adhesive layer.

11. The method of claim 7, further comprising placing at least a third patch on a bill of the headwear.

12. The method of claim 11, further comprising placing a third portion of the eyewear on a surface of the third patch.

13. A patch comprising:
a first layer; and
a second layer, wherein the first layer comprises an adhesive configured to secure the patch to a side of headwear and wherein the second layer is comprised of an elastomeric polymer configured to frictionally cling to an article that is placed on the surface thereof.

14. The patch of claim 13, wherein the article is a portion of eyewear.

15. The patch of claim 14, wherein the portion of eyewear is a stem of the eyewear.

16. The patch of claim 13, wherein the patch is in a shape of a logo.

17. The patch of claim 16, wherein the logo corresponds to the shape of sporting equipment.

18. The patch of claim 16, wherein the logo corresponds to a professional sports team logo.

19. The patch of claim 13, wherein the patch is placed on the headwear above an area between and including an ear of a person wearing the headwear and a temple of the person wearing the headwear.

20. The patch of claim 19, wherein a second, corresponding patch is placed on an opposite side of the headwear.
